(19) United States
(12) Patent Application Publication
(10) Pub. No.: US 2022/0034925 A1
(43) Pub. Date: Feb. 3, 2022

(54) GENE ANALYZER AND GENE ANALYZING SYSTEM AND METHOD USING THE SAME

(71) Applicant: Electronics and Telecommunications Research Institute, Daejeon (KR)

(72) Inventors: Kwang Hyo CHUNG, Daejeon (KR); Jin Tae KIM, Daejeon (KR); Dae Heon PARK, Daejeon (KR)

(73) Assignee: Electronics and Telecommunications Research Institute, Daejeon (KR)

(21) Appl. No.: 17/391,958

(22) Filed: Aug. 2, 2021

(30) Foreign Application Priority Data
Aug. 3, 2020 (KR) 10-2020-0096765

(51) Int. Cl. G01N 35/00; G01N 21/77; B01L 7/00

(52) U.S. Cl. CPC G01N 35/00871; G01N 21/77; B01L 7/52; G01N 2021/7786; G01N 2035/00366; G01N 2035/00455; B01L 2300/1805; B01L 2200/025; B01L 2200/028; B01L 2200/147; B01L 2300/023; B01L 2300/0609; B01L 2300/0654; G01N 2035/00881

(57) ABSTRACT

A gene analyzer has a structure in which a metal block, a heater installed on an outer surface of the metal block, and a heat insulator applied onto an outer surface of the heater are integrally formed, thereby miniaturizing the gene analyzer. Further, parts for light measurement, such as a light source element, a light-receiving element, a light source filter, a fluorescent filter, a light source lens, a fluorescent lens, and the like, are installed in a heat insulator light source path and a heat insulator fluorescent path formed in the heat insulator, and the paths formed in the heat insulator are aligned with a block light source path and a block fluorescent path of the metal block.

### CROSS-REFERENCE TO RELATED APPLICATION

This application claims priority to and the benefit of Korean Patent Application No. 10-2020-0096765 filed on Aug. 3, 2020, the disclosure of which is incorporated herein by reference in its entirety.

### BACKGROUND

### 1. Field of the Invention

The present invention relates to a technique for analyzing a gene in a sample, and more particularly, to a gene analyzer for performing isothermal gene amplification and measurement, and to a gene analyzing system and method for automatically storing in a server a result of gene analysis, using the gene analyzer.

### 2, Discussion of Related Art

In order to test a gene in a sample, a process of extracting/purifying a gene in a sample, a process of amplifying the gene, and a process of analyzing a result of amplification should be performed. The process of extracting/purifying a gene is a process of pretreatment of a sample to be analyzed and is performed through detailed operations such as sample cell lysis, binding of a gene to a solid medium, washing of a gene, and elution of a gene. Currently, gene pretreatment cartridges for the above process have been developed and automatic gene pretreatment equipment has been developed to automate the above process.

As for the process of amplifying a gene, a polymerase chain reaction (PCR) method is representatively used. In this method, amplification is performed by applying two or three steps of constant temperature cycling to a sample. As for another process of amplifying a gene, a loop-mediated isothermal amplification (LAMP) method is used. In this method, amplification is performed by maintaining a sample at a constant temperature. The LAMP method is advantageous for miniaturization of a point-of-care testing type gene analysis device because there is no need to change the temperature for gene amplification. In addition, there is an advantage in that specificity and sensitivity of analysis are improved because three or more primer sets are used for amplification.

As for the process of analyzing a result of gene amplification, a fluorescence or electrochemical microarray method, an electrophoresis method, a gel running method, a real-time fluorescence quantitative analysis method, or the like is representatively used.

Recently, equipment for performing individual processes of the above-described gene analysis and equipment for automatically performing all of the analysis processes have been developed. Further, point-of-care testing type gene analysis equipment is being developed to perform gene analysis in real time at a site where a target to be analyzed is located.

Generally, the point-of-care testing type gene analysis equipment includes a test cartridge and a gene analyzer. The test cartridge is a tool that has a portionin which a sample and a reaction solution are introduced and a gene analysis reaction is performed and is usually developed and used for a single use. The gene analyzer is usually implemented to be used exclusively for the test cartridge, is used to provide a gene reaction environment (e.g., temperature cycling, fluid control, etc.), and is used to measure a reaction result in the test cartridge and to display the reaction result as a quantitative value.

Generally, a user may check an analysis result obtained by point-of-care testing type gene analysis equipment, through a display provided on a body of a gene analyzer. In another way, the analysis result is transmitted to a mobile communication terminal such as a smartphone or the like, through a communication unit.

The above-described conventional point-of-care testing type gene analysis equipment is developed for the purpose of allowing the user to check the analysis result, and thus it is possible for the user to arbitrarily determine and process the analysis result. Therefore, the user's arbitrary intention and determination may be intervened to select and discard the analysis result, which may cause a management problem. Such a problem is particularly important when, for example, the analysis result should be managed regardless of a positive or negative analysis result in the case of point-of-care testing type gene analysis for managing infectious diseases, contagious diseases, etc. for the purpose of public interest.

### SUMMARY OF THE INVENTION

in the conventional point-of-care testing type gene analysis equipment, since a metal heating block and a fluorescence measurement module are separate, a problem of interference of a fluorescence signal occurs and a heat insulator is not applied, and thus there are limitations in temperature stabilization, low power consumption, and miniaturization of the point-of-care testing type gene analysis equipment. The present invention is directed to providing a miniaturized point-of-care testing type gene analyzer for simultaneously analyzing a plurality of analysis samples at low power.

Further, since the conventional point-of-care testing type gene analysis equipment has been developed for the purpose of allowing a user to check an analysis result, there is a possibility that a management problem may occur due to arbitrary determination and processing of the analysis result by the user. The present invention is also directed to providing a system and method for automatically storing a result of point-of-care testing type gene analysis for a sample to be analyzed in a server under the condition of authentication from a gene analyzer.

According to the present invention, there is provided a gene analyzer having a new structure for stabilizing temperature, reducing power consumption, overcoming signal interference, and miniaturization of the conventional point-of-care testing type gene analysis equipment; and a gene analyzing system and method using the gene analyzer. In order to use a loop-mediated isothermal amplification (LAMP) and measurement method, the gene analyzer includes a heating and measuring module in which a metal block, a tube insertion space, an optical path, a heater, a heat insulator, a lens, an optical filter, a mirror, a light source element, a light-receiving element, a temperature sensor, and the like are integrated.

According to one feature, there is provided a gene analyzer having a structure in which a metal block, a heater installed on an outer surface of the metal block, and a heat insulator applied onto an outer surface of the heater are integrally formed, and parts for light measurement, such as a light source element, a light-receiving element, a light source filter, a fluorescent filter, a light source lens, a fluorescent lens, and the like, are installed in a heat insulator light source path and a heat insulator fluorescent path formed in the heat insulator.

In the gene analyzer, the paths formed in the heat insulator are aligned with a block light source path and a block fluorescent path of the metal block. In the method of arranging the optical measurement parts and securing the optical paths by forming the paths in the heat insulator, when a plurality of tests are simultaneously performed using a plurality of test cartridges, light and the fluorescence may be optically separated using an adjacent test cartridge tube, thereby eliminating the signal interference problem. Further, the heat insulator aids in stabilizing the temperature by minimizing the exposure of the metal block to the outside air, thereby reducing power consumption. Further, the reduction of power consumption through the integrated structure and the heat insulator may aid in miniaturizing the gene analyzer.

According to another feature, there is provided a gene analyzing system and method that allow a result of point-of-care testing type gene analysis of a sample to be analyzed to be automatically stored in a server through a mobile communication terminal, such as a smartphone or the like, from the gene analyzer by using the gene analyzer having the above-described structure and a sample, a test cartridge, an authentication code, a printer, a mobile communication terminal, and a server which cooperate therewith.

The configurations and operations of the present invention will become more apparent from embodiments described in detail below with reference to the accompanying drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic configuration diagram of a gene analyzer according to the present invention;

FIG. 2 is a cross-sectional view of a heating and measuring module of a gene analyzer according to the present invention;

FIG. 3 is a stereoscopic view of the heating and measuring module;

FIG. 4 is an exploded view of the heating and measuring module;

FIGS. 5A to 5E are structural diagrams of an optical measuring unit of a gene analyzer;

FIG. 6 is a schematic diagram of a gene analyzing method according to the present invention;

FIG. 7 is a flowchart of a gene analyzing method according to the present invention; and

FIG. 8 is an exemplary view of a displaying content on a display of a gene analyzer.

### DETAILED DESCRIPTION OF EXEMPLARY EMBODIMENTS

Advantages and features of the present invention and methods for achieving them will be made clear from embodiments described in detail below with reference to the accompanying drawings. However, the present invention may be embodied in many different forms and should not be construed as being limited to the embodiments set forth herein. Rather, these embodiments are provided so that this disclosure will be thorough and complete and will fully convey the scope of the present invention to those of ordinary skill in the technical field to which the present invention pertains. The present invention is defined by the claims. Meanwhile, terms used herein are for the purpose of describing the embodiments and are not intended to limit the present invention. As used herein, the singular forms include the plural forms as well unless the context clearly indicates otherwise. The term “comprise” or “comprising” used herein does not preclude the presence or addition of one or more other elements, steps, operations, and/or devices other than stated elements, steps, operations, and/or devices.

Hereinafter, exemplary embodiments of the present invention will be described in detail reference to the accompanying drawings. In describing the embodiments, the detailed description of a related known configuration or function will be omitted when it obscures the gist of the present invention.

First, the concept of point-of-care testing type gene analysis according to the present invention will be briefly described. An inspector introduces a sample (bodily fluid, human tissue, an object, etc.) to be point-of-care tested into a test cartridge, and he or she inserts the test cartridge into which the sample is introduced into a gene analyzer. The gene analyzer performs preparing for gene measurement/analysis and conducting measurement/analysis. A result of the analysis may be directly checked through a display employed in the gene analyzer or may be transmitted through a communication unit,

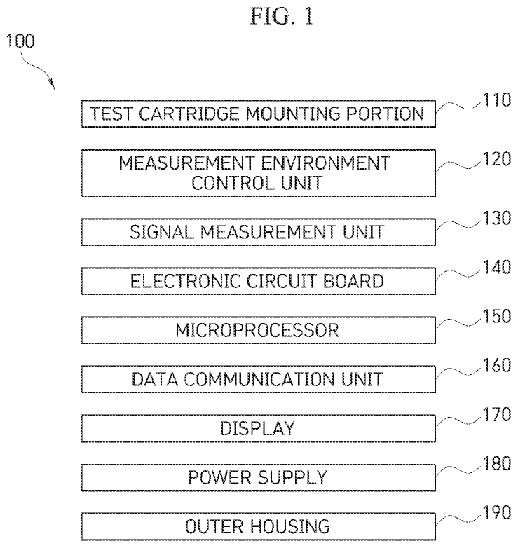

FIG. 1 illustrates components of a gene analyzer 100 used for point-of-care testing type gene analysis. The gene analyzer 100 may include a test cartridge mounting portion 110, a measurement environment control unit 120, a signal measurement unit 130, an electronic circuit board 140, a microprocessor 150, a data communication unit 160, a display 170, a power supply 180, and an outer housing 190.

The test cartridge mounting portion 110 is a component which allows an inspector to mount a test cartridge into which a sample is introduced so as to insert the sample into the gene analyzer. When the gene analyzer 100 operates in an optical measurement method, the test cartridge is preferably automatically aligned with optical measurement related parts in the gene analyzer 100 when the test cartridge is mounted, and a darkroom is formed. On the other hand, when the gene analyzer 100 operates in an electrical measurement method, an electrode in the gene analyzer 100 and the test cartridge are preferably automatically connected due to the mounting of the test cartridge.

The measurement environment control unit 120 is a unit that makes an environment for gene analysis with respect to the sample. For example, the measurement environment control unit 120 performs functions of controlling temperature, controlling an amount of light of a light source, transferring a fluid, and the like, for gene amplification. For the function of controlling the temperature, a temperature sensor, a heater, a heat insulator, a fan, a cooling fin, a metal block, and the like may be used. For the function of controlling the amount of light, a light source stabilizing electronic board, a shutter, a light source lighting time control software program, and the like may be used. For the function of transferring the fluid, a valve, a pump, a gear, and the like may be used.

The signal measurement unit 130 is a unit that measures a gene-related signal (e.g., a fluorescent signal). A light source element, an optical filter, a mirror, a lens, a light-receiving element, and the like may be used as the signal measurement unit 130.

The electronic circuit board 140 and the microprocessor 150 are units which are equipped with an analysis software program and which perform a function of controlling parts installed in the gene analyzer 100, a function of processing a measurement signal received from the signal measurement unit 130, and a function of applying an analysis algorithm. Preferably, the functions of the electronic circuit board 140 and the microprocessor 150 are integrally configured.

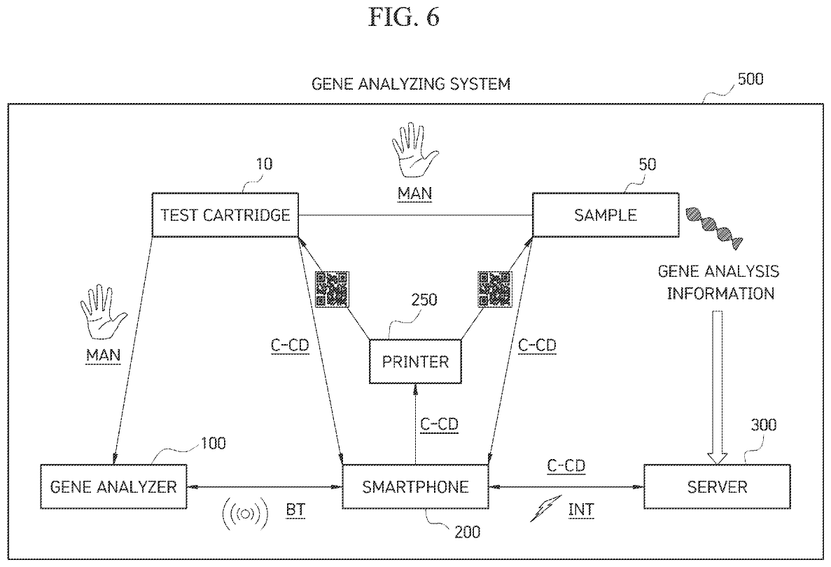

The data communication unit 160 is a component that allows the gene analyzer 100 to transmit or receive information to or from an external device, for example, a smartphone, through a Bluetooth BT communication method (see FIG. 6).

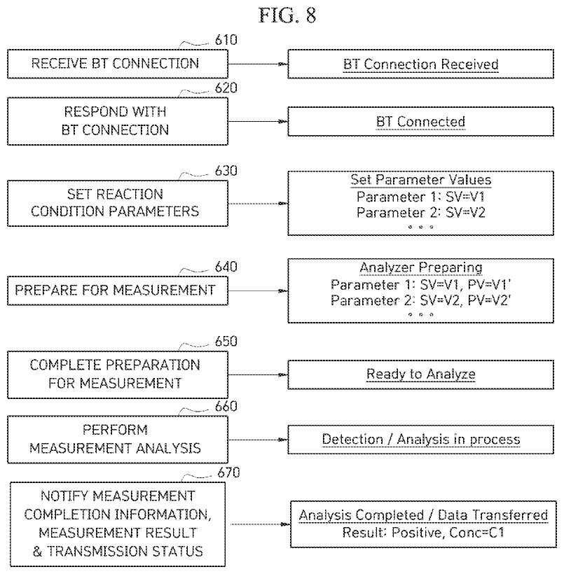

The display 170 is a component that performs a function of displaying an operating state of the gene analyzer 100, and the content displayed on the display 70 will be described below in detail h reference to FIG. 8.

The power supply 180 and the outer housing 190 are preferably reduced in size; reduced in power consumption, and miniaturized for point-of-care testing type gene analysis.

The components of the gene analyzer 100 described above may be included or omitted depending on the implementation type of the device, acrd new components may be added in accordance with the expansion of functions. Further, actual implementation parts suggested for performing the functions of the respective components may be included or omitted depending on the implementation type of the components, and new parts nay be added.

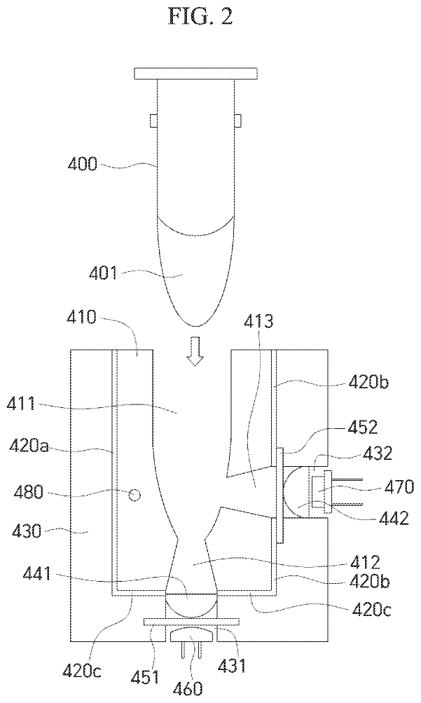

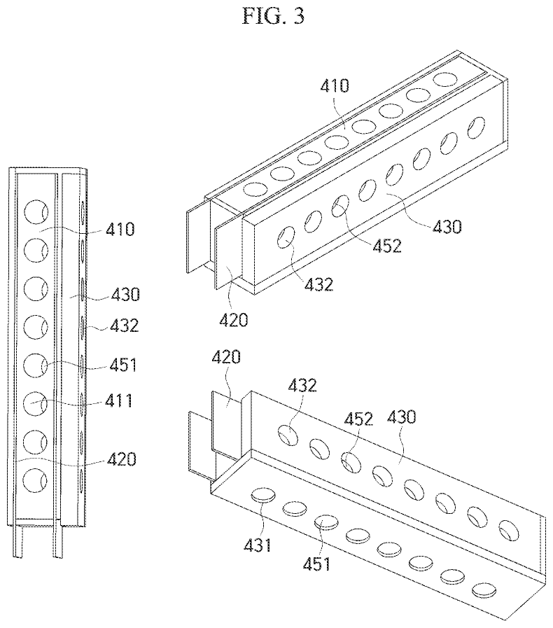

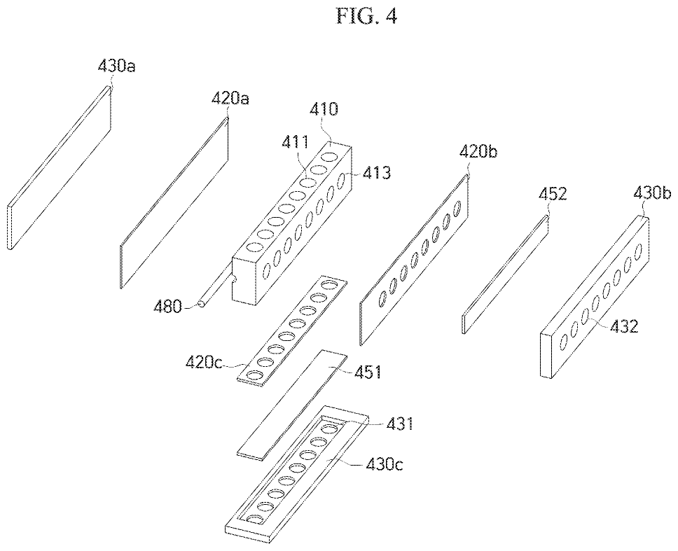

FIG. 2 is a cross-sectional view for describing an example of a configuration of a heating and measuring module which is included in the gene analyzer 100 and implemented for loop-mediated isothermal amplification (LAMP) and measurement. FIG. 3 is a stereoscopic view of the heating and measuring module illustrated in FIG. 2, and FIG. 4 is an exploded view thereof. For the LAMP and measurement, it is necessary to apply a constant temperature to a sample to be analyzed for a certain period of time and measure a fluorescence signal generated from the sample in real time. To this end, the gene analyzer 100 including the heating and measuring module as illustrated in FIG. 2 is required. Here, the heating and measuring module is a unit in which the test cartridge mounting portion 110, the measurement environment control unit 120, and the signal measurement unit 130 described above are integrally configured.

The heating and measuring module of the gene analyzer 100 according to the present invention will be described.

First, a sample to be analyzed 401 is prepared by extracting/purifying a gene and adding a reagent for gene amplification to the sample, through a sample pretreatment process. The pre-treated sample is introduced into a test cartridge tube 400. The test cartridge tube 400 is inserted into a metal block 410 in which a tube insertion space 411 capable of accommodating a tube is formed. Here, the test cartridge mounting portion 110 in FIG. 1 corresponds to the tube insertion space 411. For stable heat transfer, the tube insertion space 411 is preferably formed to be substantially the same as an external size of the test cartridge tube 400. Further, the metal block 410 is preferably formed of a metal (e.g., aluminum, an aluminum copper, a copper alloy, etc.) having high thermal conductivity in order to achieve rapid heat transfer and temperature uniformity.

In order to heat the metal block 410 at a constant temperature so that an isothermal gene amplification reaction occurs in the sample to be analyzed 401, heaters 420a, 420b, and 420c are installed on an outer surface of the metal block 410. Further a temperature sensor 480 for measuring and controlling temperature is installed inside the metal block 410. The heaters 420a, 420b, and 420c may be, for example, film heaters flexible printed circuit board (FPCB) heaters, thermoelectric elements (TEC), or the like. The temperature sensor may be, for example, a thermocouple, a resistance thermometer detector (RID), a thermistor, or the like.

The temperature of the metal block 410 is maintained at a set value by adjusting the electric power to the heaters 420a, 420b, and 420c through the electronic circuit board 140 and the microprocessor 150 in FIG. 1 on the basis of a signal of the temperature sensor 480. In order to rapidly change or maintain the temperature of the metal block 410 at a set value, the capacity of the heater is preferably sufficiently secured and the On/Off control or proportional integral derivative (PID) control is preferably performed.

Further, in order to maintain the temperature of the metal block 410 with low power and minimize the influence of the outside air, a heat insulator 430 having a predetermined thickness is applied onto outer surfaces of the metal block 410 and the heaters 420a, 420b, and 420c so that the metal block 410 and the heaters 420a, 420b, and 420c are blocked from being in direct contact with the outside air. The heat insulator may include, for example, heat insulation plastic, Styrofoam, urethane foam a glass fiber, a fiber heat insulator, a heat reflective insulating material, a vacuum heat insulator, and the like.

Meanwhile, in the sample 401 that is introduced into the test cartridge tube 400, when a specific target gene is present in the initial sample to be analyzed, the number of genes are increased due to the maintenance of a constant temperature and, accordingly, fluorescence is generated in proportion to the number of genes, and thus it is possible to determine whether the gene is amplified and to measure the amount of amplification. The fluorescent material may include, for example, FAM™, JOE™, NED™, ROX™, SYBR Green, TAMRA™, TET™, VIC®, or the like. In order to measure the fluorescence in real time, light is radiated to the sample to be analyzed 401 which is maintained at a constant temperature to measure the emitted fluorescence. To this end, a light source element 460 and a light-receiving element 470 are installed. The light source element 460 may include, for example, a light-emitting diode (LED), a laser diode (LD), or the like. The light-receiving element 470 may include, for example, a photo diode (PD), an avalanche photo diode (APD), a photo multiplier tube (PMT), or the like.

In order for efficient fluorescence emission, a light source filter 451 that transmits light in a specific wavelength band and a light source lens 441 for concentrating light on the sample to be analyzed may be additionally installed between the light source element 460 and the sample to be analyzed 401. Further, in order to selectively measure a large amount of fluorescence when fluorescence in a specific wavelength hand generated from the sample to be analyzed 401 is measured, a fluorescent filter 452 and a fluorescent lens 442 may be installed between the sample to be analyzed 401 and the light-receiving element 470.

In order to move the light passing through the sample from the light source element 460 to the light-receiving element 470, a block light source path 412 and a block fluorescent path 413 are formed in the metal block 410, and a heat insulator light source path 431 and a heat insulator fluorescent path 432 are formed in the heat insulator 430.

Further, portions of the heaters 420b and 420c installed on the optical path of the metal block 410 and overlapping the paths 413 and 412 are perforated.

The shapes and arrangement of the heater 420, the block light source path 412, the block fluorescent path 413, the heat insulator light source path 431, and the heat insulator fluorescent path 432 are configured as described above, and thus the light emission and the fluorescence measurement can be efficiently performed. Further, the optical properties, sizes, and installation positions of the light source filter 451, the light source lens 441, the fluorescent filter 452, and the fluorescent lens 442 are determined to correspond to the above configuration, and thus the configuration is preferably optimized so that the light emission and the fluorescence measurement can be efficiently performed.

Further, in order to minimize scattering, reflection, and absorption of light that may occur at an interface between a side surface of the test cartridge tube 400 and the sample to be analyzed 401, the optical components, i.e., the light source element 460, the light source filter 451, the light source lens 441, the fluorescent filter 452, the fluorescent lens 442, and the light-receiving element 470, are installed inside the block light source path 412 and the block fluorescent path 413 which are formed in the metal block 410, and inside the heat insulator light source path 431 and the heat insulator fluorescent path 432 which are formed in the heat insulator 430.

Meanwhile, for the stable measurement of the fluorescent signal, an electronic circuit for for controlling an amount of light from the light source element 460 may be installed in the electronic circuit board 140, and an electronic circuit for amplifying a signal from the light-receiving element 470 may be installed in the electronic circuit hoard 140.

As described above, the characteristic of the heating and measuring module of the present invention is that the metal block 410, the heaters 420a, 420b, and 420c installed on the outer surface of the metal block, and the heat insulator 430 installed on the outer surface of the heater are formed to have an integrated structure, and that the parts for light measurement, such as the light source element 460, the light-receiving element 470, the light source filter 451, the fluorescent filter 452, the light source lens 441, and the fluorescent lens 442, are inserted and installed inside the heat insulator light source path 431 and the heat insulator fluorescent path 432 which are installed in the heat insulator 430. Further, another characteristic of the heating and measuring module of the present invention s that the path in the heat insulator 430 is aligned with the block light source path 412 and the block fluorescent path 413 in the metal block 410. In the method of arranging the optical measurement parts and securing the optical path by forming the paths in the heat insulator 430, when a plurality of tests are simultaneously performed using a plurality of test cartridges, light and the fluorescence may be optically separated using an adjacent test cartridge tube, thereby eliminating the signal interference problem. Further, the heat insulator 430 aids in stabilizing the temperature by minimizing the exposure of the metal block 410 to the outside air, thereby reducing power consumption. Further, the reduction of power consumption owing to the heat insulator and the integrated structure aids in miniaturizing the gene analyzer 100. For reference, unlike the present invention, there is no conventional example in which a heat insulator is used, and a metal block, in which a heater is installed, and optical measurement parts are not integrated but spaced apart from each other, and thus an effect that may be obtained from the present invention may not be obtained.

Meanwhile, the number of tube insertion spaces 411, the positions and the number of the heaters 420a to 420c, and the size and arrangement of the heat insulator 430 described above may be changed as necessary. For example, in FIGS. 3 and 4, it can be seen that eight test cartridge tubes are simultaneously accommodated in one metal block 410 and three heaters 420 are installed on both side surfaces and a bottom surface of the metal block 410.

The number of the light source elements 460, the number of light-receiving elements 470, the number of light source lenses 441, and the number of fluorescent lenses 442 preferably coincide with each other so as to correspond to the number of tube insertion spaces 411, but the filters 451 and 452 may be integrated. Referring to the example of FIG. 4, the light source filter 451 and the fluorescent filter 452 are implemented as a single elongated filter so as to correspond to the eight tube insertion spaces 411 and are installed one by one. The light source lens 441 and the fluorescent lens 442 are omitted in FIG. 4.

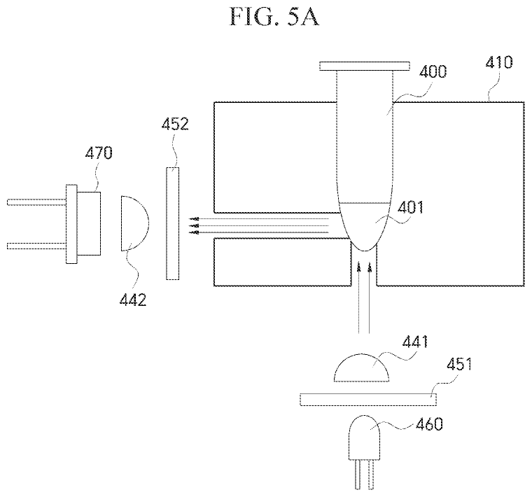

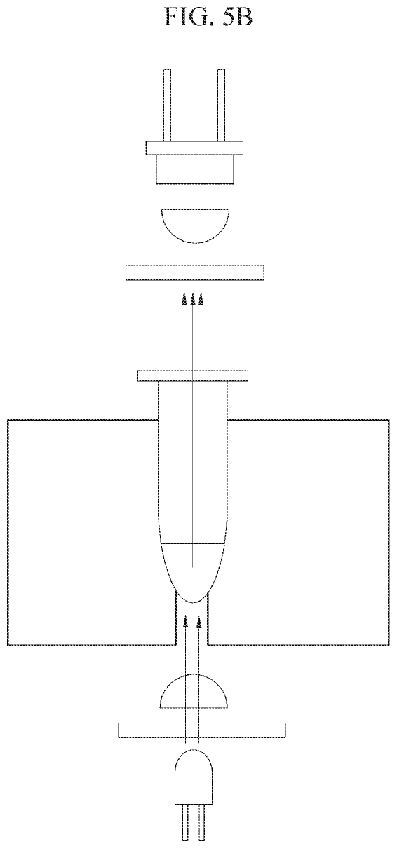

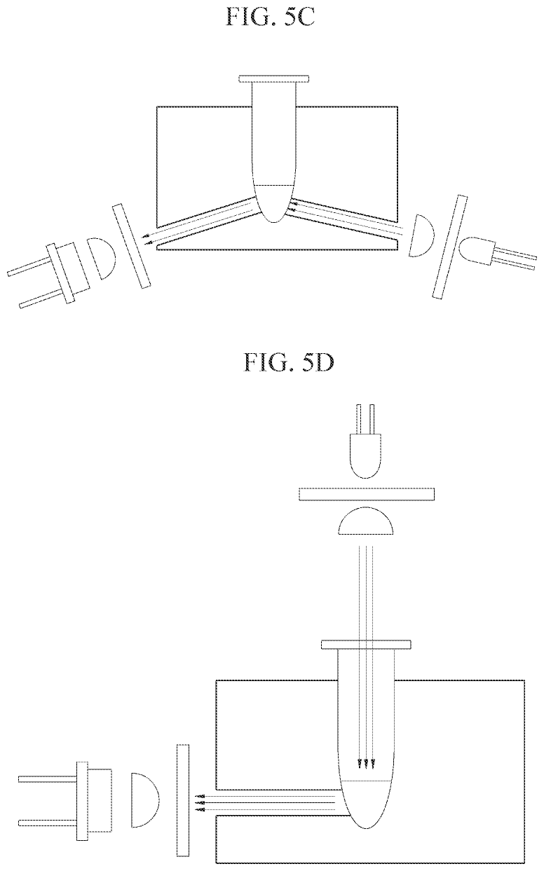

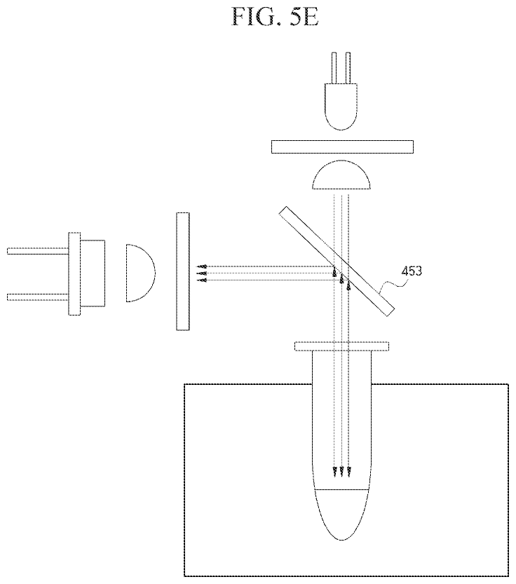

FIGS. 5A to 5E are exemplary views of various modified embodiments of an optical measurement structure of the above-described heating and measuring module. In the drawings, for convenience of description, a heat insulator 430 is omitted. FIG. 5A illustrates an optical measurement structure in the downward light emission and side-fluorescence measurement method applied to the heating and measuring module illustrated in FIG. 2. FIG. 5B illustrates an optical measurement structure in a downward light emission and upward fluorescence measurement method, FIG. 5C illustrates an optical measurement structure in a side light emission and side fluorescence measurement method, FIG. 5D illustrates an optical measurement structure in an upward light emission and side fluorescence measurement method, and FIG. 5E illustrates an optical measurement structure in an upward light emission and/or upward fluorescence measurement method. In the case of FIG. 5E, a dichroic mirror 453 may be additionally installed as necessary.

As described above, it is necessary to design the arrangement of the optical part components that may minimize a light loss due to scattering, reflection, and absorption of light that may occur at the interface between the side surface of the test cartridge tube 400 and the sample to be analyzed 401. Accordingly, it is possible to modify an optical measurement structure to an optical measurement structure not illustrated in FIGS. 5A to 5E.

Further, the shape of the tube is described in FIGS. 1 to 5, and the shape of the test cartridge may also be changed to various shapes (e.g., a flat plate cartridge in which a plurality of micro chambers and micro channels are formed) capable of accommodating the sample to be analyzed 401 and, accordingly, the optical measurement structures described above may be changed to correspond to the shape of the corresponding test cartridge.

FIG. 6 is a schematic diagram of a gene analyzing system 500 according to the present invention. A smartphone 200, which is a type of a mobile communication terminal, receives an authentication code C-CD issued for point-of-care testing type gene analysis, from a server 300 through Internet communication INT. The smartphone 200 prints the issued authentication code C-CD as a sticker in the form of a barcode or a quick response (QR) code using a printer 250. The printed sticker is attached to a sample 50 (to be precise, a sample container), which is a target to be subjected to point-of-care testing type gene analysis, and a test cartridge 10. The smartphone 200 reads the authentication code C-CD sticker, which is attached to the sample 50 and the test cartridge 10, to store analysis information. The inspector introduces the sample 50 into the test cartridge 10 by manual operation MAN and inserts the test cartridge 10 into which the sample 50 is introduced into a gene analyzer 100 by manual operation MAN. The smartphone 200 and the gene analyzer 100 are connected with each other through Bluetooth communication BT. The smartphone 200 and the gene analyzer 100 which are connected with each other exchange information related to preparing of the gene measurement/analysis, information related to conducting of the measurement/analysis, and information related to the analysis result according to the conducting of the measurement/analysis of the gene analyzer 100. Finally, the smartphone 200 which authenticates the test through the authentication code C-CD transmits the analysis information including the received analysis result to the server 300 through the Internet communication INT, and the server 300 stores the test information.

As described above, the gene analyzing system 500 according to the present invention stores the analysis information of the sample 50 and the test cartridge 10 using the authentication code C-CD and allows the gene analysis result for the sample 50 to be stored in the server 300 using the authentication code C-CD. For the above process, the smartphone 200, the gene analyzer 100, and the printer 250 are involved. It is the characteristic of the gene analysis method of the present invention that the gene analysis result of the sample 50 is automatically stored in the server 300, using the authentication code C-CD, without intervention according to the user's determination. Meanwhile, the smartphone 200 may be linked with a plurality of samples 50, the test cartridges 10, and the gene analyzers 100 to simultaneously perform a plurality of test analyses.

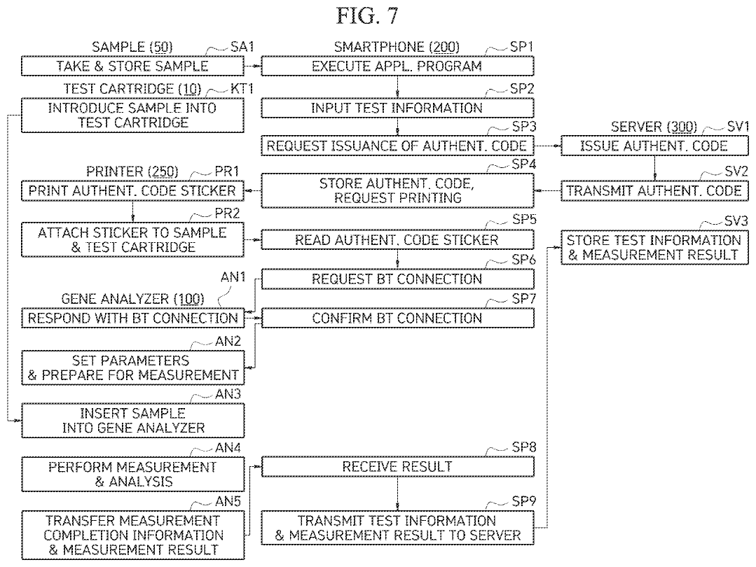

FIG. 7 is a flowchart for describing an operation sequence of the above-described gene analyzing system 500 and an information flow structure between the components of the gene analyzing system 500. FIG. 7 also shows a gene analysis method according to one aspect of the present invention,

1. Taking Sample and Inputting Test Information
SA1: an inspector takes a sample 50 and stores it in a storage container.
SP1: the inspector executes an application program of a smartphone 200.
SP2: the inspector inputs test information to the smartphone 200.

2. Issuing Authentication Code, Printing, and Authenticating
SP3: using the smartphone 200, issuance of an authentication code is requested to a server 300.
SV1: the server 300 issues the authentication code.
SV2: the server 300 transmits the authentication code to the smartphone 200.
SP4: the authentication code is stored in the smartphone 200 and a request for the authentication code output (print) is transmitted to a printer 250.
PR1: the printer 250 prints an authentication code sticker.
PR2: the inspector attaches the printed authentication code sticker to the sample container and the test cartridge 10.
SP5: the smartphone 200 reads the authentication code sticker to authenticate the sample and the test cartridge.

3. Preparing for Measurement
SP6: the smartphone 200 requests Bluetooth BT connection from a gene analyzer 100.
AN1: the gene analyzer 100 esponds with the Bluetooth connection,
SP7: the smartphone 200 confirms the Bluetooth connection.
AN2: the inspector sets reaction condition parameter values to the gene analyzer 100 to prepare for measurement.

4. Sample Introduction and Test Cartridge Insertion
KT1: the inspector introduces the sample 50 into the test cartridge 10.
AN3: the inspector inserts the test cartridge 10 into the gene analyzer 100,

5. Gene Analysis and Result Transfer and Storing
AN4: the gene analyzer 100 performs measurement and analysis,
AN5: the gene analyzer 100 transfers measurement completion information and a measurement result to the smartphone 200.
SP8: the smartphone 200 receives the measurement result from the gene analyzer 100.
SP9: the smartphone 200 transmits the test information and the measurement result to the server 300.
SV3: the server 300 stores the test information and the measurement result.

As described above, the characteristic of the gene analysis method according to the present invention is that, by using the sample 50 and the test cartridge 10 authenticated by the authentication code, the gene information of the sample is stored in the server 300 through the smartphone 200, the printer 250, and the gene analyzer 100 and, in the process, the result of the gene measurement and analysis is not affected by the inspector. To this end, the result of the gene analysis may be shown to the inspector through the gene analyzer 100 and the smartphone 200, and the result is automatically stored in the server 300 by transferring the result to the server 300 so that the result may be managed.

FIG. 8 illustrates an example of the content displayed on the display 170 (see FIG. 1) of the gene analyzer 100 according to an operation process of the gene analyzer 100. It illustrates an example in which an operation of receiving Bluetooth (BT) connection 610, an operation of responding with the Bluetooth (BT) connection 620, an operation of setting reaction condition parameters 630, an operation of preparing for measurement of gene analyzer 640, an operation of completion of the preparing for measurement 650, an operation of performing gene measurement analysis 660, and an operation of notifying measurement completion, a measurement result, and a transferred status of the result data 670, which are illustrated on the left of the drawing, may be displayed in the terms and formats as those illustrated on the right of the drawing. In addition, results of subsequent testing may be managed and reconfirmed by checking the pre-stored test information of a server 300 using a terminal (not illustrated) of the server or a smartphone 200.

According to the present invention, a point-of-care testing type gene analyzing system that overcomes the limitations of temperature stabilization, low power consumption, and miniaturization of the conventional gene analyzer and the problem of signal interference by using a heating and measuring module in which optical measurement parts are disposed in a heat insulator can be provided.

Further, the conventional point-of-care testing type gene analysis equipment has been developed for the purpose of allowing the user to rapidly check the analysis result, and thus there is a problem of management in that the analysis result can be arbitrarily interpreted and treated by a user. On the other hand, in the present invention, the result of point-of-care testing type gene analysis and the information can be automatically stored in a server without user intervention by using the authentication code, thereby increasing the reliability of point-of-care test/inspection information and overcoming the problem of management of the point-of-care testing type gene analysis.

Although the present invention has been described in detail above with reference to the exemplary embodiments, those of ordinary skill in the technical field to which the present invention pertains should be able to understand that various modifications and alterations can be made without departing from the technical spirit or essential features of the present invention. Therefore, it should be understood that the disclosed embodiments are not limiting but illustrative in all aspects. For example, although the gene analyzing system and method according to the embodiment of the present invention have been described above, the method can be applied to other measurement methods of samples. For example, the gene analyzing system and method according to the embodiment of the present invention can be applied to all immunological analysis of samples other than gene analysis. These modifications and changes in use may be made according to the purpose of users in the field, and not all of them are presented in the present embodiment.

The scope of the present invention is defined not by the above description but by the following claims, and it should be understood that all changes or modifications derived from the scope and equivalents of the claims fall within the scope of the present invention.

What is claimed is:

1. A gene analyzer that analyzes a gene-related signal from a sample introduced into a test cartridge, the gene analyzer comprising:
a metal block including a test cartridge mounting portion configured to accommodate the test cartridge and a heater configured to apply a temperature to the sample;
a heat insulator which is applied to the heater of the metal block and blocks outside air;
a light source element configured to emit light to the sample;
a light-receiving element configured to measure fluorescence generated in the sample due to the light source element;
a block light source path formed in the metal block so that the light emitted from the light source element reaches the sample;
a block fluorescent path formed in the metal block so that the fluorescence generated in the sample reaches the light-receiving element;
a heat insulator light source path formed in the heat insulator so that the light emitted from the light source element reaches the sample; and
a heat insulator fluorescent path formed in the heat insulator so that the fluorescence generated in the sample reaches the light-receiving element,
wherein the light source element and the light-receiving element are disposed in the block light source path, the block fluorescent path, the heat insulator light source path, and the heat insulator fluorescent path.

2. The gene analyzer of claim 1, further comprising, between the light source element and the sample,
a light source filter configured to transmit the light in a predetermined wavelength band from among the light emitted from the light source element; and
a light source lens configured to concentrate the light transmitted through the light source filter on the sample.

3. The gene analyzer of claim 1, further comprising, between the sample and the light-receiving element,
a fluorescent filter configured to transmit the fluorescence of in predetermined wavelength band from among the fluorescence generated in the sample; and
a fluorescent lens configured to concentrate the fluorescence transmitted through the fluorescent filter on the light-receiving element.

4. The gene analyzer of claim 1, wherein one metal block is provided and at least one test cartridge is provided.

5. The gene analyzer of claim 1, wherein the heat insulator is made of a material selected from among heat insulation plastic, Styrofoam, urethane foam, a glass fiber, a fiber heat insulator, a heat reflective insulating material, and a vacuum heat insulator.

6. The gene analyzer of claim 1, further comprising a data communication unit configured to exchange gene analysis information about the sample with an external device.

7. The gene analyzer of claim 1, further comprising a display configured to display at least one of a status of communication with an external device, setting of a reaction condition parameter for the sample, preparation and completion of gene measurement for the sample, progress of measurement and analysis, and guidance on information about completion of measurement, a measurement result, and a transmission status of the result.

8. A gene analyzing system comprising:
the gene analyzer according to claim 1;
a server configured to issue an authentication code for gene analysis;
a mobile communication terminal configured to receive the authentication code from the server;
a printer configured to print the authentication code of the mobile communication terminal;
a sample to which the printed authentication code is attached; and
a test cartridge into which the sample is introduced,
wherein the mobile communication terminal is configured to read the authentication code, which is attached to the sample and the test cartridge, to authenticate the analysis,
the gene analyzer is further configured to exchange gene analysis-related information with the mobile communication terminal,
the mobile communication terminal is configured to transmit the gene analysis-related information received from the gene analyzer to the server, and
the server is configured to store the gene analysis-related information received from the mobile communication terminal.

9. The gene analyzing system of claim 8, wherein the printer is configured to print the authentication code as a sticker in the form of one of a barcode and a quick response (QR) code.

10. The gene analyzing system of claim 8, wherein the gene analysis-related information exchanged between the gene analyzer and the mobile communication terminal includes at least one of gene measurement and analysis preparation, measurement and analysis execution, and an analysis result.

11. The gene analyzing system of claim 8, wherein the mobile communication terminal is further configured to perform gene analysis multiple times in conjunction with one or more of the gene analyzers.

12. The gene analyzing system of claim 8, wherein the mobile communication terminal is further configured to display information related to the gene analysis performed by the gene analyzer.

13. A gene analysis method performed in the gene analyzing system of claim 8, the gene analysis method comprising:
reading, by a mobile communication terminal, an authentication code, which is attached to a sample and a test cartridge, and authenticating analysis;
performing, by the gene analyzer, gene analysis and exchanging gene analysis-related information with the mobile communication terminal;
transmitting, by the mobile communication terminal, the gene analysis-related information received from the gene analyzer to a server; and
storing, by the server, the gene analysis-related information received from the mobile communication terminal.

14. The gene analysis method of claim 13, wherein a printer is configured to print the authentication code as a sticker in the form of one of a barcode and a quick response (QR) code.

15. The gene analysis method of claim 13, wherein the gene analysis-related information exchanged between the gene analyzer and the mobile communication terminal includes at least one of gene measurement and analysis preparation, measurement and analysis execution, and an analysis result.

16. The gene analysis method of claim 13, wherein the mobile communication terminal is further configured to perform gene analysis multiple times in conjunction with one or more of the gene analyzers.

17. The gene analysis method of claim 13, wherein the mobile communication terminal is further configured to display information related to the gene analysis performed by the gene analyzer.
